(19) United States
(12) Patent Application Publication
(10) Pub. No.: US 2011/0082306 A1
(43) Pub. Date: Apr. 7, 2011

(54) BIRCH REDUCTION OF STEROID SUBSTRATES VIA ALKALI METAL - SILICA GEL MATERIALS

(76) Inventors: Michael J. Costanzo, Ivyland, PA (US); Mitul N. Patel, Union, NJ (US); Paul F. Vogt, Albany, NY (US)

(21) Appl. No.: 12/933,971

(22) PCT Filed: Mar. 24, 2009
(86) PCT No.: PCT/US09/38070

§ 371 Date: Dec. 20, 2010

Related U.S. Application Data
(60) Provisional application No. 61/038,847, filed on Mar. 24, 2008.

(51) Int. Cl. C07J 51/00

(52) U.S. Cl. 552/505

(57) ABSTRACT

Steroids are well known, medicinal relevant, chemical entities. There are numerous steroids which have fully or partially reduced ‘A’ rings. These fully or partially reduced ‘A’ rings have been reduced in the past through Birch and other dissolving metal reductions. Novel reducing conditions for steroid substrates are described which utilizes stabilized alkali metal-silica gel materials. The invention relates to a method for reducing a double bond within a steroid by contacting an unsaturated steroid having a phenyl ring with a Stage 0 or Stage I alkali metal-silica gel material in the presence of a proton source under reaction conditions sufficient to form a reduced steroid having a diene structure.

### CROSS REFERENCE TO RELATED APPLICATION

This application claims priority to PCT International Application No. PCT/US2009/038070, filed Mar. 24, 2009, which claims priority to U.S. Provisional Application No. 61/038,847, filed Mar. 24, 2008, which is incorporated herein by reference.

### BACKGROUND OF THE INVENTION

Steroids are well known medicinal entities that possess the skeleton of cyclopentanoperhydrophenanthrene (1). They include a wide range of naturally occurring compounds among which are the sterols, the sex hormones, the adrenocorticoid hormones, the cardiac glycosides and vitamin D.

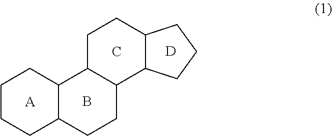

There are numerous steroids that have fully or partially reduced ‘A’ rings. These fully or partially reduced ‘A’ rings have been reduced in the past through Birch and other dissolving metal reductions; for a review see H. Pellissier and M. Santelli in Org. Prep. Proced. Int. 2002, 34(6), 611-642. Despite its great utility in the synthesis of steroids, the classic Birch reduction has several undesirable attributes that have limited its use, particularly on a large scale. Primary among these are safety concerns associated with the high toxicity of liquid ammonia, the hazards of cryogenic temperatures (−30° C. and below), and the well known dangers of handling metallic alkali metals. Clearly, there is a need to improve reaction conditions for the Birch reduction of unsaturated steroids.

### SUMMARY OF THE INVENTION

The invention relates to a method for reducing a double bond within a steroid by contacting an unsaturated steroid having a phenyl ring with a Stage 0 or Stage I alkali metal-silica gel material in the presence of a proton source under reaction conditions sufficient to form a reduced steroid having a diene structure.

### DETAILED DESCRIPTION OF THE INVENTION

Steroids are a group of polycyclic compounds which include cholesterol, numerous hormones, precursors of certain vitamins, biles acids, alcohols, (sterols), and various drugs. Steroids have a common fused, 17-carbon ring system, cyclopentanoperhydrophenanthrene (1). Any steroid having the basic skeleton of cyclopentanoperhydrophenanthrene (1) above where ring A is a phenyl ring may be reduced by a reduction method of the invention. Such unsaturated steroids have the 17 carbon skeleton shown in formula (2). As is known in the art, different steroids have a variety of substituents on the 17-carbon skeletons of formulas (1) and (2). The Stage 0 or Stage I alkali metal-silica gel materials used in the invention are free flowing solids that reduce the dangers typically associated with the handling of alkali metals, resulting in a safer modification of the classic Birch reduction that completely avoids the use of liquid ammonia and can avoid the use of cryogenic temperatures.

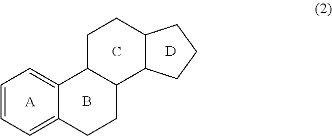

Most steroids have two methyl groups and an aliphatic side chain of 2 to 10 carbon atoms, more often 8-10 carbon atoms, except estrogens and androgens which may not have the aliphatic side chain. Scheme 1, below, shows the reduction of a double bond within a steroid according to the invention. A steroid, such as shown below, is contacted with a Stage 0 or Stage I alkali metal-silica gel material in the presence of a homogeneous or heterogeneous proton source under reaction conditions sufficient to form the corresponding reduced steroid. The reactions are generally run at sub-ambient to ambient temperatures with or without the presence of either an alkylamine or alkyldiamine additive. The “A” ring of the steroid may be selectively reduced by the method of the invention.

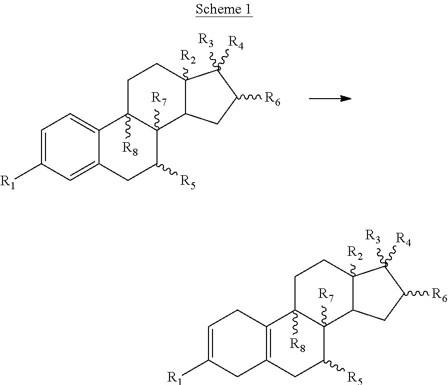

While any steroid may be reduced using the invention, in this exemplary reaction, R₁ can be a C₁ to C₁₀ alkoxy, C₂ to C₆ alkynyloxy, aryloxy, C₁ to C₁₀ alkylsilyloxy, aryl-C₁ to C₁₀-alkylsilyloxy. Preferably R₁ is methoxy, ethoxy, phenyloxy, trimethylsilyloxy, t-butyldimethylsilyloxy, t-butyldiphenylsilyloxy. R₂, R₆, R₇, and R₈ can each independently be hydrogen or C₁ to C₁₀ alkyl and each is preferably hydrogen or methyl. R₃ can be hydrogen, hydroxyl, C₁ to C₁₀alkoxy, C₁ to C₁₀ alkyl, C₂ to C₁₀ alkynyl, C₁ to C₁₀ alkylsilyloxy, aryl-C₁ to C₁₋₁₀-alkylsilyloxy, or a cyclic ketal. R₃ is preferably hydrogen, hydroxyl, methyl, methoxy, ethoxy, trimethylsilyloxy, t-butyldimethylsilyloxy, t-butyldiphenylsilyloxy, R₄ can be hydrogen, hydroxyl, C₁ to C₁₀ alkyl or taken together with R₃ to make a cyclic ketal such as ethylene ketal, propyplene ketal and 2,2-dimethylpropylene ketal. Preferably, R₄ is hydrogen, hydroxyl, or methyl. R₆ can be hydrogen or hydroxyl. Other examples of steroid compounds which can be reduced using a method of the invention are described by H. Pellissier and M. Santelli in Org. Prep. Proced. Int. 2002, 34(6), 611-642.

The Alkali Metal-Silica Gel Material

Alkali metals are those metals in the Group 1 family of the periodic table, and are known to have limited uses in organic synthesis owing to their pyrophoric character in presence of trace amount of moisture. Chemists used these metals as such for Wurtz couplings, acyloin condensations, and other reactions or by dissolving them in liquid ammonia to accomplish the otherwise difficult reduction of aromatics (Birch reduction) and other compounds. The terms “Group 1 metal” or “Group 1 metals” are used here to describe alkali metals and alloys of alkali metals. The alkali metals include lithium (Li), sodium (Na), potassium (K), rubidium (Rb), and cesium (Cs).

Recently, new alkali metal-silica gel materials having improved handling and safety characteristics have been described. These new materials have an alkali metal or alkali metal alloy absorbed into silica gel. The new materials retain the reactivity of the native metal, while being much less dangerous than the bulk metal. Accordingly, the term “alkali metal-silica gel material” as used herein refers to the material that is formed when an alkali metal, or an alkali metal alloy, is absorbed into porous silica gel. The different types of alkali metal-silica gel materials, and the process of making the material, are described in detail in U.S. Published Patent Application No. 20050151278, filed Nov. 24, 2004 and published Jul. 14, 2005, which is entitled “SILICA GEL COMPOSITIONS CONTAINING ALKALI METALS AND ALKALI METAL ALLOYS.” This application is incorporated herein by reference. The alkali metal-silica gel materials are available from SiGNa Chemistry, New York, N.Y. (www.signachem.com), Alfa Aesar (www.alfa.com), or Sigma-Aldrich (www.sigmaaldrich.com). The alkali metal-silica gel materials are optimally loaded with 35 to 40 wt. % alkali metal, but lower loadings are also possible.

As is disclosed in U.S. Published Patent Application No. 20050151278, given the pyrophoric nature of alkali metals and their alloys, the ability to utilize alkali metals or their equivalents in a convenient form continues to be a need in the chemical industry. However, the stability of alkali metals and alkali metal alloys in air can be dramatically improved by absorbing the alkali metals into porous silica gel. For example, these metals can be made significantly more stable by absorption into silica gel to form the alkali metal-silica gel materials. In terms of newer process development this idea was attractive owing to its operational simplicity; as such, solid-state reducing agents could in principle be employed in a fixed bed flow reactor, potentially replacing the traditional stirred batch mode of doing chemical reactions.

The alkali metal-silica gel materials are described in U.S. Published Patent Application No. 20050151278 with reference to Stages 0, I, II, or III. The stages differ in their preparation and chemical reactivity, and each successive stage may be prepared directly or from an earlier stage material. Preferred alkali metal-silica gel materials are those containing sodium, potassium, or sodium-potassium alloys with sodium and sodium-potassium alloys being most preferred. Stage 0 and Stage I alkali metal-silica gel materials (described below) are useful in this invention.

The Stage 0 alkali metal-silica gel material is a loose black powder that retains much of the reducing ability of the alkali metals. This material is prepared by contacting an alkali metal or alkali metal alloy with silica gel under isothermal conditions, preferably at or just above room temperature. The Stage 0 materials are pyrophoric but less dangerous in air as compared to their parent Group 1 metal.

More specifically, the Stage 0 material is a Group 1 metal/silica gel composition comprising the product of mixing a liquid Group 1 metal, such as Na, or a liquid Group 1 metal alloy, such as K₂Na or Na₂K, with silica gel under isothermal conditions sufficient to absorb the liquid Group 1 metal or liquid Group 1 metal alloy into the silica gel pores. Preferred Group 1 metals for Stage 0 materials include a low-melting Group 1 metal such as cesium or a NaK alloy. The Stage 0 Group 1 metal/silica gel composition reacts with dry O₂, which differentiates it from Stage I, II, and III materials. Since Stage 0 material is reactive with dry air, it should be handled in vacuo, in an oxygen-free atmosphere, and preferably in an inert atmosphere, such as under nitrogen or an inert gas.

To form Stage 0 materials, a Group 1 metal is mixed with silica gel in an inert atmosphere under isothermal conditions, preferably at room temperature or slightly above, for a time sufficient to permit the alkali metal or alloy to be absorbed into the silica. The mixing must be done in an inert atmosphere such as within a glove box or glove bag. During formation of a preferred Stage 0 material, a liquid Group 1 metal, such as Na₂K, may be poured over a bed of silica gel at room temperature. The mixture is agitated, preferably stirred or shaken, to achieve good mixing. The liquid Group 1 metal is preferably absorbed into the porous silica gel.

Depending upon the Group 1 metal used, the absorption of the liquid alloy of Group 1 metals in silica form Stage 0 material preferably occurs within 15° C. of room temperature (25° C.). In the typical process, the sample converts to a product which is a free-flowing amorphous black powder, in which the individual particles have a shiny surface. The mixture is agitated for a time sufficient to allow the alkali metal or alloy to be absorbed or “soaked up” by the silica gel. The time of mixing generally depends upon the batch size of material being prepared and may range from several minutes to several hours.

When preparing Stage 0 material, any heat generated by the reaction or put into the reaction should be controlled or dissipated. A significant temperature increase during the preparation should be avoided, as it may result in the formation of Stage I material. The temperature may be controlled by spreading the silica gel (for example, on a metal tray), stirring the silica gel, and/or by cooling the reaction vessel. The reaction temperature should, however, be maintained such that the Group 1 metal remains liquid so that it may be absorbed by the silica gel.

The Stage 0 material is a shiny black powder that reacts exothermically with water. While the exact composition of the Stage 0 material is not currently known, Stage 0 materials exhibit endothermal processes at temperatures which are lower that the melting point of the most common Group 1 alloys, such as NaK, thus indicating that small particles of the Group 1 alloys are within the pores of the silica gel.

The Stage 0 materials are the most reactive members of the alkali metal-silica gel materials. Since the addition of a low-melting alkali metal or alloy to silica gel produces a Stage 0 material without significant heat evolution, the Stage 0 material retains most of the reducing ability of the alkali metal. Because of their reactivity toward air and moisture they must be handled with care and not allowed to come in contact with large amounts of air and, especially, moisture.

The Stage I alkali metal-silica gel material is a loose black powder that is indefinitely stable in dry air, and is the product of mixing a liquid Group 1 metal with silica gel under exothermic conditions sufficient to absorb the liquid Group 1 metal into the silica gel pores. The resulting material does not react with dry O₂.

The Stage I alkali metal-silica gel material may be formed by mixing the liquid Group 1 metal, at or just above its melting point with silica gel under an inert atmosphere to allow the Group 1 metal to be absorbed into the pores of the silica gel. The Group 1 metal may also be mixed with the silica gel using one of the alternative methods discussed above, such as adding the Group 1 metal as a vapor. The mixture is then maintained at or slightly above the melting point of the Group 1 metal (i.e., approximately 70° C. to 150° C.) and agitated for between several minutes to several hours. Generally speaking, higher reaction temperatures convert the material in shorter times. The reaction to form Stage I materials is mildly exothermic, and, on a large scale, the process would be preferably done by adding the liquid metal or alloy to the silica gel in a metal pan that would remove heat as it is produced. The reaction appears to form an alkali metal-silica gel lattice. The exothermic nature of the reaction differentiates Stage I material from Stage 0 material. Heating above the exotherm can convert Stage I material to Stage II or Stage III material, depending upon the temperature. U.S. Patent Application Publication No. 20050151278, which is noted above, describes Stage 0, I, II, and III materials in detail.

The simplest and most direct preparation of Stage I materials is to heat Stage 0 samples overnight under an inert atmosphere at temperatures of 140° C. Other times and temperatures may work also, but care should be taken to avoid overheating, which can lead to the formation of Stage 11. To insure a homogeneous product, provision should be made for agitation during the heating process.

The Stage I material is an amorphous black powder that does not immediately react with dry air, but reacts exothermically with water. The difference between Stages I and 0 is that the former can be handled in dry air and even quickly transferred in ordinary laboratory air without catching fire or degrading rapidly. When kept under an atmosphere of dry oxygen for hours to days, Stage I material (in contrast to Stage 0 material which reacts with dry O₂) is unchanged and produces the same amount of hydrogen gas upon reaction with liquid water as do fresh samples.

The properties of the Stage 0 and Stage I alkali metal-silica gel materials are summarized in Table 1 below.

TABLE 1
Summary of Stages 0 and I

| Material | | |
|---|---|---|
| Type | Preferred Metals/Alloys Used | Preparation Procedures |
| Stage 0 | Liquid alloys (NaK, Na₂K, K₂Na, etc.) | Under inert atmosphere or vacuum, liquid |
| | | alkali metal alloy is added to silica gel at or |
| | | near room temperature. On a large-scale, |
| | | this process would be best done by adding |
| | | the liquid metal or alloy to silica gel spread in |
| | | a metal pan that would dissipate any heat |
| | | which may be produced. |
| Stage I | Liquid alloys (NaK, Na₂K, K₂Na, etc.) | Under inert atmosphere or vacuum, solid, |
| | Neat sodium, potassium, cesium, | liquid or vapor alkali metal is added to silica |
| | rubidium, etc. | gel and is agitated for several minutes to |
| | | hours at a temperature of about 150° C. |

The preferred Stage 0 and Stage I alkali metal-silica gel materials include 35-40 wt % alkali metal or alkali metal alloy on silica gel. For Stage 0, K₂Na and Na₂K are the preferred alkali metals. For Stage I, Na, K, NaK, Na₂K, and K₂Na are the preferred alkali metals.

The stoichiometry of a Birch reduction requires 2 moles of alkali metal (1 reaction equivalent) per mole of double bond reduced in the substrate; i.e. a molar ratio of 2:1. The preferred molar ratios of alkali metal to double bond reduced in the substrate in the invention are from 2:1 to 40:1 (1 to 20 reaction equivalents). The particularly preferred molar ratios of alkali metal to double bond reduced are from 16:1 to 32:1 (8 to 16 reaction equivalents).

The Proton Sources

An exemplary method of the invention involves contacting the respective functionalized steroid with an alkali metal-silica gel material in the presence of a homogeneous or heterogeneous proton source under conditions sufficient to form the corresponding reduced steroid. The presence of the proton source is important, as it facilitates the reaction. In intimate physical mixtures with the alkali metal-silica gel materials, proton sources were found to enable protonation of the radical anion, anion, and/or dianion intermediates.

A suitable proton source may be either homogenous (completely soluble) or heterogenous (partially soluble) under the reaction conditions. In addition, the proton source should not react with the alkali metal-silica gel material at a rate that is competitive with protonation of the anion intermediates, which includes radical anions, dianions and other anion intermediate species. For example, the proton source may be an alcohol capable of protonating alkali metal carbanion salt. In addition, other characteristics of prospective proton sources such as ionization constant (pKₐ), solvent polarity, solubility, and kinetics of proton transfer should be considered while choosing a suitable proton source. For example, it is preferred that the proton source be easy to handle and separate from the product, be able to protonate the generated anion intermediates, and be unreactive, or only slowly reactive, toward the absorbed alkali metal. Furthermore, a preferred proton source will have a fast kinetics of proton transfer to the anion intermediates.

Suitable homogenous proton sources include, but are not limited to, alcohols such as ethanol, isopropanol, sec-butanol, tert-butanol and 2-methyl-2-butanol. Suitable heterogenous proton sources include, but are not limited to, (NH₄)₂HPO₄, NaH₂PO₄, NH₄Cl, KHP (potassium hydrogen phthalate), and NaHCO₃. Other mild, organic soluble proton sources, such as weak acids, may also be suitable proton sources.

The stoichiometry of a Birch reduction requires 2 moles of proton (1 reaction equivalent) per mole of double bond reduced in the substrate; i.e. a molar ratio of 2:1. The preferred molar ratios of alkali metal to double bond reduced in the substrate in the invention are from 2:1 to 40:1 (1 to 20 reaction equivalents). The particularly preferred molar ratios of alkali metal to double bond reduced are from 15:1 to 30:1 (7.5 to 15 reaction equivalents).

Amine Additives

For certain steroid substrates it may be desirable to add either ammonia, an amine, or a diamine to the reaction mixture in order to facilitate the formation the corresponding reduced steroid product and/or to prevent unwanted side reactions. For example, addition of either propylamine or ethylenediamine either minimizes or prevents unwanted dealkylation of arylalkyl ethers to the corresponding phenols. In addition, the presence of alkyldiamines, such as ethylenediamine, can allow the Birch reduction to be conducted at more practical temperature than would otherwise be permissible; for example −5° C. vs. −40° C. The amines and alkyldiamines include, but are not limited to, unsubstituted and alkyl-substituted compounds such as methylamine, ethylamine, n-propylamine, n-butylamine n-pentylamine, ethylenediamine, N-methylethylenediamine, N,N′-dimethylethylenediamine, N,N,N′,N′-tetramethylethylenediamine, 1,3-diaminopropane, 1,4-diaminobutane, etc.

The preferred molar ratios of the amine/diamine additive per mole of alkali metal are from 1 to 10 moles of amine/diamine additive per mole of alkali metal; i.e. molar ratios of 1:1 to 10:1. The particularly preferred molar ratios of amine/diamine additive per mole of alkali metal are from 1 to 6 moles of amine/diamine additive per mole of alkali metal; i.e. molar ratios of 1:1 to 6:1.

Solvents

The solvent for the reactions described herein may be any suitable organic aprotic solvent. In addition, mixtures of aprotic solvents, including those with different polarities as well as with crown ethers may also be used. Because the alkali metal-silica gel material can react with protons to form H₂ in the reaction, it is necessary that the solvent should not exchange protons with the reaction materials. Suitable nonpolar aprotic solvents include, for example, hydrocarbons such as heptane, cyclohexane and toluene whereas suitable polar aprotic solvents include ethers such as tetrahydrofuran (THF). It is preferred that the reactions be carried out in an inert gas atmosphere with dried solvents under anhydrous conditions.

Polar aprotic solvents, such as THF, are particularly suitable because they can provide reasonable solubilities of the reactants, intermediates and products, and they can be easily removed from the reaction products. Other suitable polar aprotic solvents include diethylether, methyl tert-butyl ether (MTBE), 1,2-dimethoxyethane (DME), diethyleneglycol dimethyl ether, 2-methyltetrahydrofuran, 1,4-dioxane and hexamethylphosphoric acid triamide (HMPA). Certain protic solvents, such as alcohols may serve as a solvent/cosolvent provided that the reduction is conducted at a temperature low enough, such as −60° C. or less, to slow down the rate of reaction of the protic solvent with the alkali metal-silica gel material.

Reaction Chemistry

As described herein, alkali metal-silica gel materials can be used to carry out the Birch reduction of steroid substrates either in the presence of suitable proton sources or by subsequent addition of soluble proton sources or other electrophiles. In order to facilitate an effective reaction, various reaction conditions should be satisfied.

For example, it is preferred that the pKₐ of a homogeneous proton source be lower than that of the substrate. However, the pKa of the proton source should not be too low (pKa<16) because the proton source may react with alkali metals, especially at temperatures greater than −60° C., to give off hydrogen rather than delivering it to the anionic species generated in medium. The preferred pKₐ range for heterogeneous solid proton sources is 8-10. Polar aprotic solvents, such as THF or other dry ethers, are particularly suitable solvents for heterogeneous solid proton sources. Protic solvents such as water, methanol or ethanol may be used after the reaction is complete to quench any remaining alkali metal-silica gel material. Furthermore, while any molar ratio between the alkali metal and the steroid substrate will effect some Birch reductions, it is preferred that the molar ratio of the alkali metal to the steroid substrate be greater than two to drive the reaction efficiently to completion.

Furthermore, while this reaction works well at sub-ambient temperatures, unlike other methods that require harsher reaction conditions, adjusting the temperature may maximize the stoichiometric efficiency of the process. Preferably, according to the invention, a steroid to be reduced is contacted with a Stage 0 or I alkali metal-silica gel material in the presence of a homogeneous or heterogeneous proton source at a reaction temperature ranging from about −60° C. to about 25° C., and more preferably from about −10° C. to about 5° C. However, with either alkali metal-silica gel material, as it is desired to maximize the yield of the Birch reduction, it may also be desirable facilitate the reaction, by increasing the temperature.

In addition, it should be noted that the reactions may be conducted under microwave irradiation, which may accelerate some reactions in moderately conducting solvents. However, using this method may cause the metals to spark because of the exposure to the microwave irradiation, and to overheat, which means that one has to find the proper conditions for the microwave-assisted reductions.

Suitable Reaction Processes

The methods of the invention may be carried out using various industrial reaction processes. For example, the reactions of the invention may be carried out in batch or fixed-bed flow reaction conditions, with each having satisfactory results. As will be understood by a person of ordinary skill in the art, batch process reactors are the simplest type of reactor. A batch reaction process consists of filling the reaction vessel with the desired reaction components, and allowing the reaction to proceed, typically with stirring to promote contact and mixing of the reagents under specific desired reaction conditions. At the conclusion of the reaction, the reaction mixture is removed from the reactor and subjected to physical (filtration) and chemical (e.g. solvent evaporation, crystallization, chromatography) separation steps to isolate desired products, and the process may be repeated. With respect to the invention, a batch process may be used to contact the chosen solid Stage 0 or Stage I alkali metal-silica gel and proton source materials with a steroid, and then allowing the reaction to proceed under conditions sufficient to complete the reaction and form the corresponding reduced steroid. Alternatively, the proton source or other electrophile may be withheld until the reduction is complete and then added in a subsequent step.

With continuous process reactors, or continuous flow reactors, fresh reaction materials are continuously added to the reactor and the reaction products are continuously removed. As a result, the material being processed continuously receives fresh medium and products and waste products and materials are continuously removed for processing. Advantages of using a continuous process reactor are numerous. For example, the reactor can thus be operated for long periods of time without having to be shut down, thereby resulting in the continuous process reactor being be many times more productive than a batch reactor. An example of a continuous process reactor is a fixed-bed flow reactor in which a liquid solution of reaction substrate is percolated through a column of solid reagent, such as alkali metal-silica gel, with direct collection of the product solution at the column's exit. For sequential reactions, the soluble proton source or other electrophile may be present in the receiving flask. While virtually any type of reaction process and reactor may be used for the reactions described herein, a continuous process reactor, such as a fixed-bed flow column reactor, is the preferred reactor type for the reactions of the invention.

### EXAMPLES

### Example 1

### Reduction of (1,1-Dimethylethyl)-[[(17β)-3-methoxyestra-1,3,5(10)-trien-17-yl]oxy]dimethylsilane with K₂Na-SG(I) in the Presence of Ethylenediamine

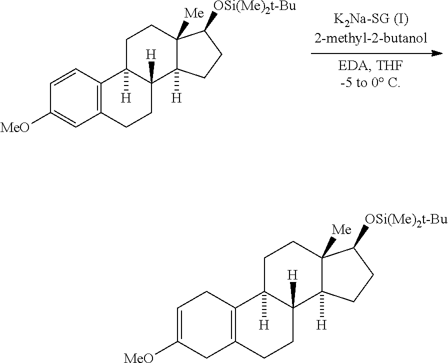

Stage I K₂Na-silica gel (K₂—Na-SG(I)) (33.6 g, 0.116 mol; 35.1 wt % K₂Na) was weighed into a 3-neck round-bottomed flask in a glove box under argon, which was subsequently fitted with a glass-paddle mechanical stirrer and 2 rubber septa. The reaction vessel was then transferred to a fume hood and a nitrogen inlet needle and a digital thermometer were inserted through the septa. Anhydrous THF (40 mL) was added via syringe and the resulting slurry was mechanically stirred under nitrogen and cooled to −5° C. with a chiller. Ethylenediamine (25 mL, 0.374 mol; EDA) was added followed by the slow addition of 2-methyl-2-butanol (25 mL, 0.229 mol) over 25 minutes while not allowing the temperature to exceed 10° C. A solution of (1,1-dimethylethyl)-[[(17β)-3-methoxyestra-1,3,5(10)-trien-17-yl]oxy]dimethylsilane (5.00 g, 0.0125 mol; CAS# 113507-13-4) in THF (10 mL) was added and the resulting mixture was stirred at −5 to 0° C. After 18 hours, the reaction mixture was partially quenched by the cautious addition of methanol (50 mL) at 0-20° C. under nitrogen, stirred for 1 hour at 0-5° C., and then fully quenched by the dropwise addition of water (10 mL) at 5-20° C. under nitrogen. After stirring at 0-5° C. for 1 hour, the insoluble material was removed by filtration and the filtrate was extracted twice with 50 mL portions of ethyl acetate. The combined ethyl acetate layers were dried over sodium sulfate and concentrated in vacuo to yield crude product as a waxy solid. The crude product was recrystallized twice from ethanol to afford [[(17β)-3-methoxy-estra-2,5(10)-dien-17-yl]oxy]-1,1-dimethylethyl)-dimethylsilane as a white flocculent crystalline solid (3.28 g, 65%): mp 112-114° C. (uncor); ¹H NMR (500 MHz, CDCl₃) [symbol] 14.66-4.62 (m, 1H), 3.59 (t, J=8.3 Hz, 1H), 3.54 (s, 3H), 2.95-2.81 (m, 1H), 2.72-2.44 (m, 3H), 2.14-1.99 (m, 1H), 1.94-1.79 (m, 3H), 1.79-1.77 (m, 1H), 1.73-1.66 (m, 1H), 1.64-1.53 (m, 2H), 1.49-1.40 (m, 1H), 1.39-1.21 (m, 3H), 1.21-1.09 (m, 1H), 1.09-0.98 (m, 2H), 0.87 (s, 9H), 0.72 (s, 3H), 0.01 (s, 3H), 0.0 (s, 3H); EI-MS m/z 402 (M.)⁺

### Example 2

### Reduction of (1,1-Dimethylethyl)-[[(17β)-3-methoxyestra-1,3,5(10)-trien-17-yl]oxy]dimethylsilane with K₂Na-SG(I) in the Presence of n-Propylamine

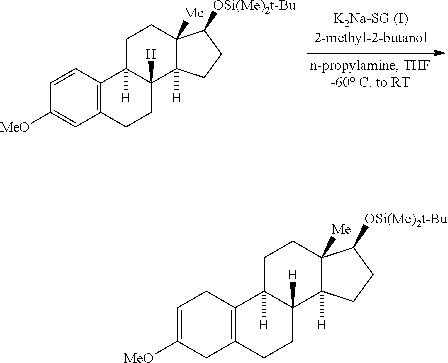

Stage I K₂Na-silica gel (K₂—Na-SG(I)) (462 mg, 1.17 mmol; 25.2 wt % K₂Na) was weighed into a 1-neck round-bottomed flask in a glove box under argon, which is subsequently fitted with a glass-magnetic stirrer and a rubber septa. The reaction vessel was then transferred to a fume hood and a nitrogen inlet needle and a digital thermometer were inserted through the septa. Anhydrous n-propylamine (1.1 mL, 13.4 mmol) was added via syringe and the resulting slurry was cooled to −60° C. while stirring under nitrogen. A solution of (1,1-dimethylethyl)-[[(17β)-3-methoxyestra-1,3,5(10)-trien-17-yl]oxy]dimethylsilane (100 mg, 0.250 mmol; CAS# 113507-13-4) in mixture of THF (0.4 mL) and 2-methyl-2-butanol (0.4 mL) was added dropwise over 10 min at −57 to −60° C. The resulting mixture was allowed to slowly warm to room temperature over 18 h. The reaction mixture was partially quenched by the cautious addition of methanol (1 mL) at 5° C. while under nitrogen and then fully quenched by the dropwise addition of water (1 mL) at 5° C. The reaction mixture was extracted twice with 5 mL portions of ethyl acetate. The combined ethyl acetate layers were washed with brine dried over sodium sulfate and concentrated in vacuo to yield crude product as a waxy solid. The crude product was purified via preparative TLC eluting on silica gel with heptane/ethyl acetate (19:1) afford [[(17β)-3-methoxy-estra-2,5(10)-dien-17-yl]oxy]-1,1-dimethylethyl)-dimethylsilane as a white solid (34 mg, 34%).

### Example 3

### Reduction of (1,1-Dimethylethyl)-[[(17β)-3-methoxyestra-1,3,5(10)-trien-17-yl]oxy]dimethylsilane with K₂Na-SG(I) in the Presence of a 5:1 Mixture of THF and 2-Methyl-2-butanol

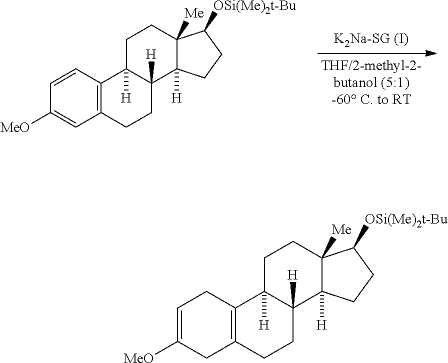

Stage I K₂Na-silica gel (K₂-Na-SG(I)) (936 mg, 2.33 mmol; 25.2 wt % K₂Na) was weighed into a 1-neck round-bottomed flask in a glove box under argon, which is subsequently fitted with a glass-magnetic stirrer and a rubber septa. The reaction vessel was then transferred to a fume hood and a nitrogen inlet needle and a digital thermometer were inserted through the septa. A pre-cooled solution of 2 mL of a mixture of THF and 2-methyl-2-butanol (5:1) was added via syringe and the resulting slurry was cooled to −60° C. A solution of (1,1-dimethylethyl)-R(17β)-3-methoxyestra-1,3,5(10)-trien-17-yl]oxy]dimethylsilane (100 mg, 0.250 mmol; CAS# 113507-13-4) in 3 mL of a mixture of THF and 2-methyl-2-butanol (5:1) was added dropwise to the reaction mixture over 10 min at −60° C. The resulting mixture was allowed to slowly warm to room temperature over 18 h. The reaction mixture was partially quenched by the cautious addition of methanol (1 mL) at 5° C. while stirring under nitrogen and then fully quenched by the dropwise addition of water (1 mL) at 5° C. The reaction mixture was extracted twice with 5 mL portions of ethyl acetate. The combined ethyl acetate layers were washed with brine and dried over sodium sulfate and concentrated in vacuo to yield crude [[(17β)-3-methoxy-estra-2,5(10)-dien-17-yl]oxy]-1,1-dimethylethyl)-dimethylsilane as a waxy solid (95 mg, 95% yield).

### Example 4

### Reduction of (1,1-Dimethylethyl)-[[(17β)-3-methoxyestra-1,3,5(10)-trien-17-yl]oxy]dimethylsilane with Na-SG(I) in the Presence of Ethylenediamine

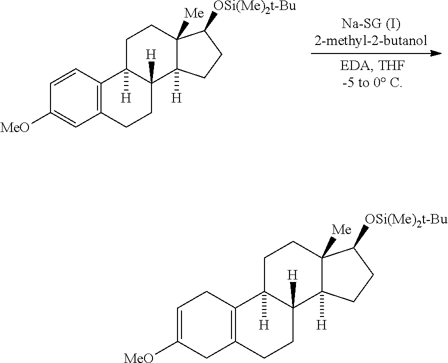

Stage I Na-silica gel (Na-SG(I)) (33.9 g, 0.399 mol; 27.1 wt % Na) was weighed into a 3-neck round-bottomed flask in a glove box under argon, which was subsequently fitted with a glass-paddle mechanical stirrer and 2 rubber septa. The reaction vessel was then transferred to a fume hood and a nitrogen inlet needle and a digital thermometer were inserted through the septa. Anhydrous THF (40 mL) was added via syringe and the resulting slurry was mechanically stirred under nitrogen and cooled to −5° C. with a chiller. Ethylenediamine (25 mL, 0.374 mol; EDA) was added followed by the slow addition of 2-methyl-2-butanol (27 mL, 0.399 mol) over 25 minutes while not allowing the temperature to exceed 10° C. A solution of (1,1-dimethylethyl)-[[(17β)-3-methoxyestra-1,3,5(10)-trien-17-yl]oxy]dimethylsilane (5.00 g, 0.0125 mol; CAS# 113507-13-4) in THF (10 mL) was added and the resulting mixture was stirred at −5 to 0° C. After 24 hours, the reaction mixture was partially quenched by the cautious addition of methanol (50 mL) at 0-20° C. under nitrogen, stirred for 1 hour at 0-5° C., and then fully quenched by the dropwise addition of water (10 mL) at 5-20° C. under nitrogen. After stirring at 0-5° C. for 1 hour, the insoluble material was removed by filtration and the filtrate was extracted twice with 50 mL portions of ethyl acetate. The combined ethyl acetate layers were dried over sodium sulfate and concentrated in vacuo to yield crude [[(17β)-3-methoxy-estra-2,5(10)-dien-17-yl]oxy]-1,1-dimethylethyl)-dimethylsilane as a waxy solid.

The claimed invention is:

1. A method for reducing a double bond within a steroid by contacting an unsaturated steroid having a phenyl ring with a Stage 0 or Stage I alkali metal-silica gel material in the presence of a proton source under reaction conditions sufficient to form a reduced steroid having a diene structure.

2. A method of claim 1, wherein the unsaturated steroid has the 17 carbon skeleton of formula (2) (2).

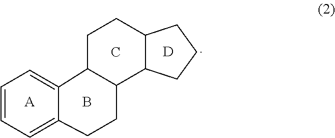

3. A method of claim 2, wherein the unsaturated steroid is reacted with a Stage 0 alkali metal-silica gel material.

4. A method of claim 3, wherein the alkali metal is a NaK alloy.

5. A method of claim 2, wherein the unsaturated steroid is reacted with a Stage 1 alkali metal-silica gel material.

6. A method of claim 5, wherein the alkali metal is Na, K, or a NaK alloy.

7. A method of claim 2, 3, or 5 wherein the protein source is an alcohol.

8. A method of claim 2, 3 or 5 wherein the unsaturated steroid is contacted with the Stage 0 or Stage I alkali metal-silica gel material in the presence of an amine or a diamine.

9. A method of claim 2, 3, or 5 wherein the unsaturated steroid and the Stage 0 or Stage I alkali metal-silica gel material are contacted in an aprotic solvent or a mixture of aprotic solvents.

10. A method of claim 8, wherein the polar aprotic solvent is THF.
